(19) United States
(12) Patent Application Publication
(10) Pub. No.: US 2025/0057689 A1
(43) Pub. Date: Feb. 20, 2025

(54) SYSTEMS AND METHODS FOR REGULATING TEMPERATURES AROUND EXTREMITIES

(71) Applicant: ParachuteAdams LLC, Rochester, MN (US)

(72) Inventors: Fredric B. Meyer, Rochester, MN (US); Mohamed Bydon, Rochester, MN (US); Tobias Prothers Van Schaick, Rochester, MN (US); Preksha Gupta, Rochester, MN (US); Jacob Raul Lovett Mohamed, Rochester, MN (US); Wallid Taaha Soukaki, Rochester, MN (US); Kevin Yang, Rochester, MN (US)

(73) Assignee: ParachuteAdams LLC, Rochester, MN (US)

(21) Appl. No.: 18/719,233

(22) PCT Filed: Dec. 1, 2022
(86) PCT No.: PCT/US2022/051522

§ 371 Date: Jun. 12, 2024

Related U.S. Application Data
(60) Provisional application No. 63/289,763, filed on Dec. 15, 2021.
(60) Provisional application No. 63/335,812, filed on Apr. 28, 2022.

(51) Int. Cl. A61F 7/00; A61G 10/02; A61M 11/00

(52) U.S. Cl. CPC A61F 7/0053; A61G 10/02; A61M 11/00; A61F 2007/0045; A61F 2007/0064; A61F 2007/0076; A61F 2007/0086; A61F 2007/0096; A61G 2210/70

(57) ABSTRACT

The present disclosure generally relates to systems and methods for regulating temperatures around an extremity of a subject. For example, the subject may be a subject having neuropathy, or other conditions in which control of the temperature around the extremity may be beneficial. For example, one aspect is generally directed to a device for regulating temperatures. The device can be used to surround an extremity, such as a hand or foot. The device may include a frame in which the extremity is located, and optionally fabric or other material draped around at least portion of the frame. These may be positioned so as to avoid contact with the extremity. Air may be distributed in an interior region within the frame, e.g., to regulate the temperature therein. In addition, in some cases, aromatic substances such as essential oils may be present in the interior region. Other aspects are generally directed to regulating the temperature of an extremity of a subject, e.g., a subject having neuropathy or other conditions. In some cases, this may be controlled by the subject. Yet other aspects are directed to methods of making such devices, kits involving such devices, or the like.

### RELATED APPLICATIONS

This application claims the benefit of U.S. Provisional Patent Application Ser. No. 63/289,763, filed Dec. 15, 2021, entitled “Systems and Methods for Regulating Temperatures around Extremities”; and of U.S. Provisional Patent Application Ser. No. 63/335,812, filed Apr. 28, 2022, entitled “Systems and Methods for Regulating Temperatures around Extremities.” Each of these is incorporated herein by reference in its entirety.

### FIELD

The present disclosure generally relates to systems and methods for regulating temperatures around an extremity of a subject. For example, the subject may be a subject having neuropathy, or other conditions in which control of the temperature around the extremity may be beneficial.

### BACKGROUND

Some subjects suffer from painful lower extremity pain, and such pain may be more intense at night, e.g., when the patient is lying in bed. Such pain may be caused by a variety of conditions, including neuropathies such as sensory degenerative peripheral neuropathy, restless legs, causalgia, diabetes, chemotherapy, trauma, burns, fractures, post infection, phantom limb, etc. This pain is often described as “burning,” and can prevent subjects from sleeping well at night. Often, the hands or feet are hypersensitive, and even bed sheets lightly touching those extremities can cause discomfort or pain. The condition may be more frequent in the elderly, who can sometimes become cognitively impaired due to the effects of chronic sleep deprivation.

It is estimated that up to 15% of American adults have some sort of restless legs syndrome and that 20 million have peripheral neuropathy of all types, e.g., involving the feet. There is a large cottage industry of unproven medications and physical manipulations that are most often ineffective and oftentimes expensive. Accordingly, improvements are needed.

### SUMMARY

The present disclosure generally relates to systems and methods for regulating temperatures around an extremity of a subject. For example, the subject may be a subject having neuropathy, or having other conditions in which control of the temperature around the extremity may be beneficial. The subject matter of the present disclosure involves, in some cases, interrelated products, alternative solutions to a particular problem, and/or a plurality of different uses of one or more systems and/or articles.

One aspect is generally directed to a device for regulating temperature around an extremity of a subject. In one embodiment, the device comprises a structure comprising a shaped frame defining an interior region having a volume at least sufficient to contain an extremity of a subject, a cooling system able to produce cooled air, and a distributor positioned to distribute the cooled air from the cooling system into the interior region of the shaped frame. In some cases, the volume is no more than 10 ft³.

In another embodiment, the device comprises a structure comprising a shaped frame defining an interior region, a brace immobilizing the shaped frame on a mattress or a bedframe, a cooling system able to produce cooled air, and a distributor for distributing the cooled air from the cooling system into the interior region of the shaped frame.

The device, in yet another embodiment, comprises a structure comprising a shaped frame defining an interior region, a stand positioning the shaped frame at a height of between 1.5 ft and 3 ft over a bottom region of the stand, a cooling system able to produce cooled air, and a distributor for distributing the cooled air from the cooling system into the interior region of the shaped frame.

Another aspect is generally directed to a method for regulating temperature around an extremity of a subject. In one embodiment, the method comprises providing a structure comprising a shaped frame defining an interior region, a fabric covering at least portion of the interior region, and a distributor positioned to distribute air into the interior region of the shaped frame; positioning the structure and the subject relative to each other such that the subject has at least one extremity positioned within the interior region, where the at least one extremity does not contact the fabric, and the shaped frame does not surround an entire limb of the subject; producing cooled air in a cooling system; and distributing the cooled air at the extremity within the interior region of the shaped frame using the distributor.

The method, in another embodiment, comprises providing a structure comprising a shaped frame defining an interior region and having a fabric covering; positioning the structure and the subject relative to each other such that the subject has at least one extremity positioned within the interior region, wherein the at least one extremity does not contact the fabric, and the shaped frame does not surround an entire limb of the subject; and distributing cooled air at the extremity within the interior region of the shaped frame using a distributor immobilized relative to the shaped frame.

In another aspect, the present disclosure encompasses methods of making one or more of the embodiments described herein, for example, a device for regulating temperatures. In still another aspect, the present disclosure encompasses methods of using one or more of the embodiments described herein, for example, a device for regulating temperatures.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIGS. 1A-1B illustrate an example device in accordance with one embodiment;

FIGS. 2A-2D illustrate the use of various devices in other embodiments.

FIG. 3 illustrates various shaped frames, in accordance with certain embodiments;

FIG. 4 illustrates various distributors, in other embodiments; and

FIG. 5 illustrates additional shaped frames, in still other embodiments.

### DETAILED DESCRIPTION

The present disclosure generally relates to systems and methods for regulating temperatures around an extremity of a subject. For example, the subject may be a subject having neuropathy, or other conditions in which control of the temperature around the extremity may be beneficial. For example, one aspect is generally directed to a device for regulating temperatures. The device can be used to surround an extremity, such as a hand or foot. The device may include a frame in which the extremity is located, and optionally fabric or other material draped around at least portion of the frame. These may be positioned so as to avoid contact with the extremity. Air may be distributed in an interior region within the frame, e.g., to regulate the temperature therein. In addition, in some cases, aromatic substances such as essential oils may be present in the interior region. Other aspects are generally directed to regulating the temperature of an extremity of a subject, e.g., a subject having neuropathy or other conditions. In some cases, this may be controlled by the subject. Yet other aspects are directed to methods of making such devices, kits involving such devices, or the like.

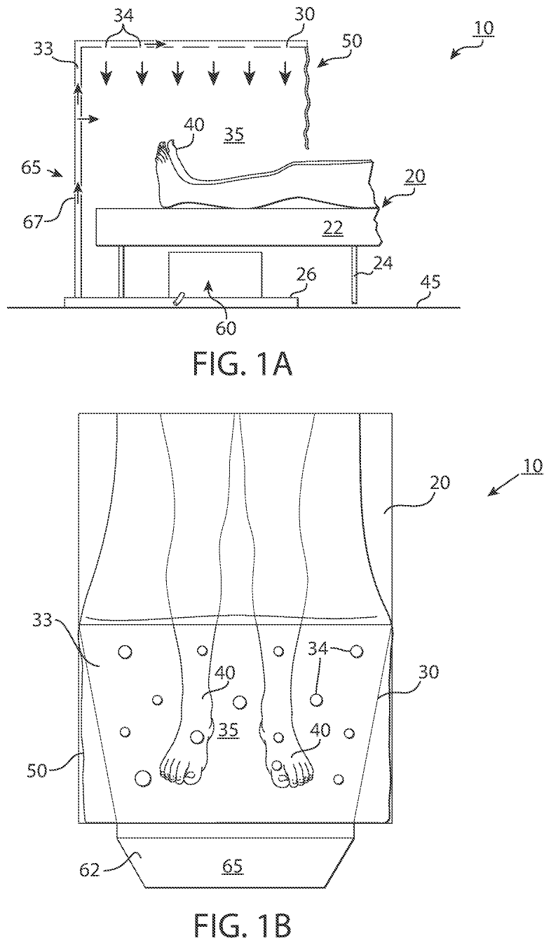

One non-limiting example of a device in accordance with one aspect of the present disclosure is shown in FIGS. 1A and 1B. In these figures, a device 10 for regulating the temperature around an extremity is shown. In this figure, device 10 is positioned near a bed 20, which includes one or more mattresses 22 and a bedframe 24 supporting the mattresses. The bed may also include pillows, sheets, blankets, or other bedding materials typically associated with a bed (not shown here for purposes of clarity). However, it should be understood that the bedframe is optional. The device may, for example, be placed on the bed, immobilized relative to the bed (e.g., immobilized relative to the mattress or the bedframe, etc.), placed or mounted on the floor, etc. In FIG. 1, device 10 is shown placed on floor 45, with a base 26 that is positioned under bedframe 24. However, this is by way of example only, and in other embodiments, other techniques for positioning the device near the bed are also possible. In addition, although bed 20 is shown in this figure, in other embodiments, the device may be affixed or positioned near other suitable articles of furniture, e.g., a couch or a sofa.

Device 10 in FIG. 1A may also have a frame 30 defining an interior region 35. The frame, in some cases, may be a shaped frame, e.g., having a relatively solid shape, such as is shown with frame 30 in FIG. 1A. For example, the frame may be composed of relatively rigid components, which serve to define an interior space, e.g., the shaped frame is sufficiently solid that it supports its own weight and defines an interior space. For instance, the frame may comprise plastic, metal (e.g., aluminum), fiberboard, or other materials. In some cases, the frame may be adjustable. In addition, in some embodiments, the frame may be a non-shaped frame, i.e., the frame need not have a specific or predetermined shape, but may instead be amorphous or flexible, etc.

In some cases, the frame may define an interior region that is generally sized to contain an extremity (e.g., a foot or a hand). For instance, in FIG. 1A, the feet 40 of a subject are present within interior region 35. In some embodiments, the interior region may be sized to allow the temperature therein to be controlled or regulated. For example, in some cases, the volume of the interior region may be relatively small, e.g., less than 10 ft³ (0.28 m³) or less than 5 ft³ (0.14 m³), or other volumes or dimensions such has those described herein. However, in another embodiment, the interior region may be sufficiently large so as to contain therein the entire subject, or the entire subject except for the head, or an entire limb of a subject (e.g., an arm or a leg), etc.

In addition, in certain embodiments, the interior region may be at least partially enclosed using one or more fabrics or other materials. These may be attached directly to the frame, or not attached and placed on top of the frame. For example, a frame may be shaped to keep the fabrics or other materials from substantially entering the interior region. In some cases, the fabric or other materials may define an at least partially enclosed volume containing one or more extremities, e.g., based on the interior region of the frame. These may fully enclose the frame, e.g., to define the interior region, or there may be one or more openings in some cases, e.g., such that the limbs of the subject can extend out of the interior region (for example, in embodiments where the interior region is sized to contain an extremity of a person, but not the whole of the person). As a non-limiting example, in FIG. 1A, fabric 50 is placed on top of frame 30, and portions may hang in front and/or the sides to at least partially enclose interior region 35.

In one set of embodiments, one or more portions of a subject, such as a hand or a foot, may be positioned within the interior region such that the portions does not come into contact with any part of the device (other than the bedding or mattress that the subject is laying on). For example, the subject's extremity may not physically contact the frame, fabric, source of air, or other portions of the device. In contrast, various prior art systems include blankets or other bedding materials that typically contact the subject, including the extremities. Such contact can be uncomfortable for certain subjects, e.g., those for whom may be sensitive to contact, or wounds on an extremity, or other conditions such as those described herein, etc. However, in other embodiments, the subject may come into contact with various parts of the device, such as the fabric, frame, etc. For example, as is shown in FIG. 1A, the feet 40 of the subject are positioned within interior region 35, but are not in contact with fabric 50 or frame 30. However, it should be understood that the subject remains in contact with portions of bed 20 (including mattress 22, or common bedding materials such as pillows, sheets, blankets, etc.).

Air may be distributed into the interior region using a variety of techniques. For example, a distributor may be used to direct air into the interior region from one or more locations. The distributor may include components such as conduits, manifolds, tubes, ducts, pipes, or the like. The distributor may be integrated within the frame of the device, and/or form separate components of the device. For example, in FIG. 1A, the distributor includes one or more ducts 33 having a plurality of openings 34 through which air may be distributed into interior region 35.

In some embodiments, the air may be produced by a suitable source of air. The source of air may draw air into the device, and optionally, heat and/or cool the air to control its temperature, for example, using thermoelectric coolers and/or heaters (e.g., Peltier modules) or any other cooling and/or heating systems such as any of those described herein. As an example, as is shown in FIG. 1A, source of air 60 may produce air, which passes through conduit 65 to be distributed as noted above. Air flows within conduit 65 as indicated by arrows 67. In this figure, source of air 60 is present on base 26 of device 10, but it should be understood that this is by way of example only, and other configurations are also possible. For example, the source of air may be separate from the frame, or there may be a separate distributor that is not part of the frame, etc. Other configurations are described in more detail below.

As mentioned, in some cases, the source of air may also heat or cool the air to be distributed in the interior region, e.g., to control the temperature within the interior region. For example, the source of air may include an air conditioner, an electric heater, or other heating or cooling systems such as those described herein. In addition, in some cases, the source of air may include both a heating system and a cooling system.

In addition, in some embodiments, aromatic substances such as essential oils, fragrances, perfumes, or the like may be introduced into the air, e.g., for distribution in the interior region. Such aromatic substances may be added, for example, for various aromatherapeutic applications. These may be added, for example, to soothe or calm the subject. In some cases, for example, air may be directed over a suitable source of aromatics, e.g., a solid block or a container containing a liquid, for example, such that the aromatic substances can become dispersed or distributed into air from the source of air. As another non-limiting example, an aromatic substance may be nebulized for introduction into the air, e.g., for distribution in the interior region.

In some embodiments, the device may optionally include one or more temperature sensors to determine the temperature within the interior region. These may be in any suitable location, e.g., located within or attached to the frame or the distributor, or present as a separate component, etc. In addition, in some cases, the air entering the device may be controlled, at least in part, based on such temperatures, e.g., to ensure that the temperature within the interior region is at or near a specific value or setpoint, or within a certain range, etc. Such temperatures may be predetermined, or controlled by a user of the device, etc. In some cases, the device may adjust the amount of heating or cooling, and/or the flow rate at which air enters the interior region, based on such temperatures.

The subject may be any subject. Typically, the subject is a human. The subject may be a normal subject, or one with a condition such as neuropathy (e.g., sensory degenerative peripheral neuropathy). In some cases, the subject may be a subject that is hypersensitive or experiences heightened sensitivity. For instance, contact with an external object, such as a blanket, may be experienced as discomfort, irritation, pain, tingling, itching, crawling, a pins-and-needles sensation, etc. As another example, the subject may be one who has an injury or a wound, e.g., on an extremity, and it may be desirable to avoid or at least minimize contact with external objects to promote healing and/or avoid irritation, infection, etc.

FIG. 1B illustrates a top view of the device shown in FIG. 1A. In this figure, the feet 40 of a subject lying in bed 20 are shown. Frame 30 of device 10 is elevated above the subject's feet via vertical leg 62, containing conduit 65. While only a single vertical leg is shown in this figure, this is by way of example only, and in other embodiments, more than one vertical leg may be present as part of frame 30. In addition, covering frame 30 is fabric 50.

Air flows upwardly through conduit 65 into the upper portion of frame 30 to be distributed by duct 33, having a plurality of openings 34, acting as a distributor of the air into interior region 35. Duct 33, in this example is relatively large and generally trapezoidal shaped. However, in other embodiments, the duct may have different shapes, and/or more than one duct may be present. In addition, in some embodiments, no duct may be present and other methods may be used to distribute air.

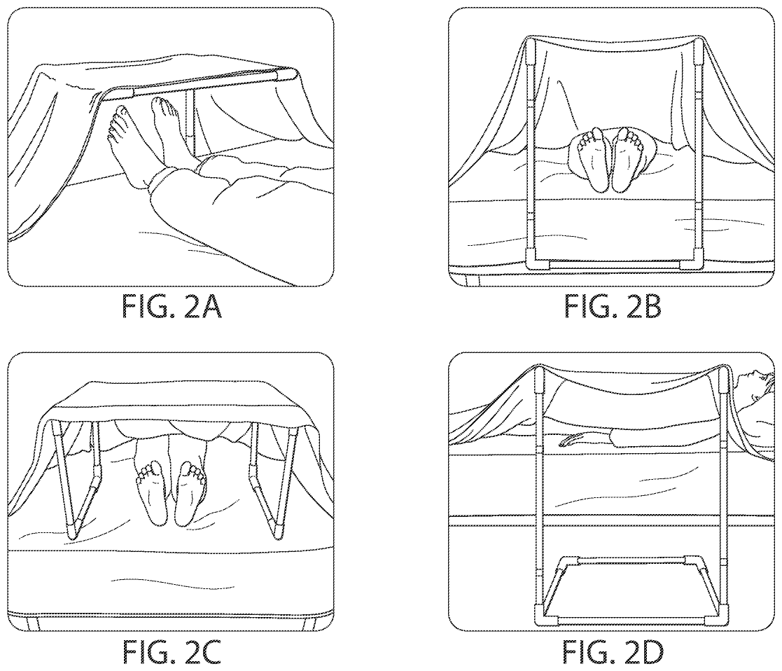

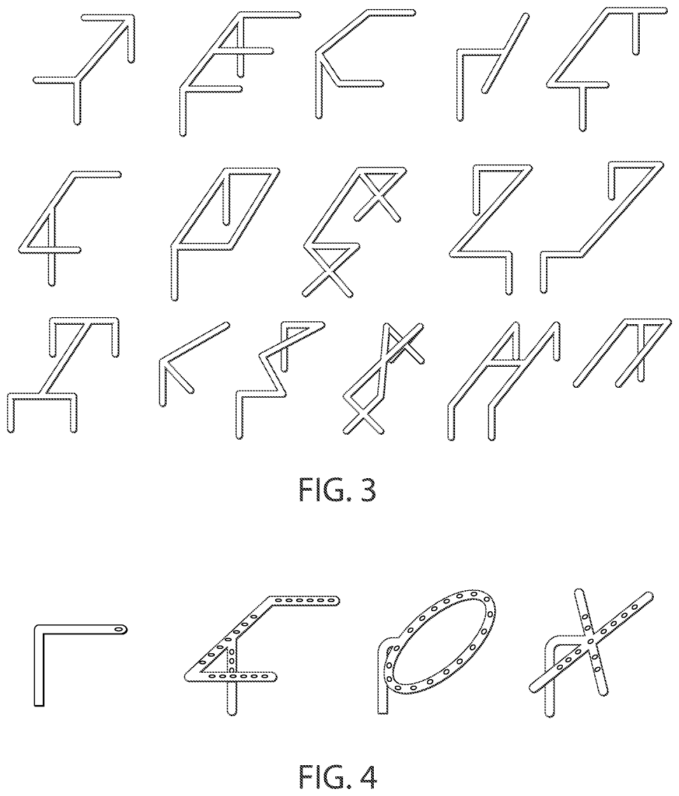

In addition, various specific non-limiting examples of such devices are shown in FIG. 2. For example, FIG. 2A illustrates a device having a shaped frame formed from a single vertical leg and an upper platform that supports a fabric, thereby defining a partially covered interior region under the upper platform. A subject's feet are positioned within the interior region.

FIG. 2B illustrates another device that is partially immobilized between a mattress and a bedframe of a bed. The shaped frame includes two arms extending upwardly from between the mattress and the bedframe. These arms are connected to horizontal poles at right angles, which in turn are connected by a lateral pole, forming a square U shape over and substantially parallel to the mattress. This U shape can be used to support a fabric, and defines an interior region, into which a subject's feet are positioned.

Yet another device is shown in FIG. 2C. In this example, the device is placed on the bed, and is not immobilized relative to the bed. Instead, the device stays in position due to its own weight. The shaped frame in this example includes two U-shaped legs, each end of the leg supporting a corner of a horizontal substrate positioned substantially to the mattress, in the manner of a table. The horizontal substrate is used to support a fabric, and defines an interior region between the substrate and the surface of the bed, in which a subject's feet can be positioned.

Still another device is shown in FIG. 2D. In this example, the device includes a stand positioned on the floor (i.e., under the bed), and two vertical legs that form the shaped frame, which extend upwardly from the stand. These legs are connected to horizontal poles at right angles, which in turn are connected by a lateral pole, forming a square U shape over and substantially parallel to the mattress. This is similar to the device shown in FIG. 2B. However, in this example, the device is not immobilized relative to the bed, and does not substantially rely on the bed for support. Instead, the upper portion of the frame is positioned over the bed, forming an internal region between the shaped frame and the surface of the bed. In this example, the internal region is used to contain a subject's hand and arm.

Accordingly, FIGS. 1 and 2 illustrate various non-limiting examples of different embodiments of the present invention. However, other embodiments are also possible in addition to the examples provided above. Thus, more generally, various aspects of the invention are directed to various systems and methods for regulating temperatures around an extremity of a subject, as discussed in more detail below.

For instance, certain aspects are generally directed to systems and methods of regulating temperatures around an extremity of a subject. The subject may be lying, resting, sleeping, etc. on a bed, couch, or sofa, etc., and may be conscious or unconscious. The subject may be a human, and may be healthy or have a condition, such as a medical condition.

As a non-limiting example, the subject may be one in which contact with an object is uncomfortable or unpleasant; for instance, the subject may be hypersensitive or experiences heightened sensitivity. For example, contact with an external object may be experienced by such subjects as pain, discomfort, irritation, tingling, itching, crawling, a pins-and-needles sensation, etc. For such subjects, it would be desirable to avoid or at least minimize any contact with external objects, since even mild forms of physical contact can cause discomfort or pain, etc. Without wishing to be bound by any theory, this may be caused by conditions such as neuropathy (e.g., sensory degenerative peripheral neuropathy), allodynia, neuralgia, restless legs, causalgia, diabetes, chemotherapy, trauma, burns, fractures, post infection, phantom limb, traumatic injuries, infections, metabolic problems, toxins, inherited or genetic causes toxins, etc. In some cases, the cause is not known.

As another example, the subject may be one that has suffered an injury, and the injury site should be protected to avoid or minimize contact with external objects, e.g., to facilitate or promote the healing process. For example, the injury may be an open wound, or a wound covered with a bandage, and avoiding contacting the wound with external objects may be important to promote healing, minimize the risk of infection, prevent or limit pain sensations, interfering with the bandage or other equipment, or the like. As another example, the subject may have an arm or a leg in a cast.

As yet another example, the subject may have equipment, such as medical equipment that has been attached or inserted into the subject. For instance, equipment such as casts, IV or other tubing, electrodes (e.g., EKG electrodes), etc., may be present, and it may be desirable to avoid or minimize contact of that equipment to external objects. As still another example, the subject may have a tattoo, paint, prosthetics, or other applications that have been applied, to which contact should be minimized or avoided. In another example, the subject may simply desire to minimize or avoid physically contacting an extremity for various personal reasons.

Thus, in certain embodiments, it may be desired to avoid contact with one or more extremities (e.g., the hands or feet), or other portions of the subject (for example, an arm, leg, thigh, etc.), while regulating temperatures around an extremity of a subject, for example, if the subject is lying, resting, sleeping, etc. In some cases, only a single portion of the subject may be protected to avoid or minimize contact with external objects (for example, a single extremity, such as a hand or a foot), although in other cases, more than one such portion may be protected (e.g., both of a person's feet, the feet of two different subjects, etc.). In addition, in some embodiments, the temperature of an entire limb of a subject (e.g., an arm or a leg) may be regulated, although in other embodiments, only the temperature around the extremity itself is regulated.

In one set of embodiments, a portion of a subject, e.g., one or more extremities, may be partially or completely contained within an interior volume that is defined, in whole or in part, by a frame of the device. The frame, in some embodiments, may be a shaped frame, e.g., a frame having a relatively solid or predefined shape. For example, in some cases, the shaped frame may be composed of relatively rigid components that together define an interior volume and a shape. The shaped frame may thus be sufficiently solid that the shaped frame defines a shape that is not substantially altered (e.g., sags, becomes permanently deformed, or loses its shape or its interior volume, etc. due to gravity or other forces). For example, the shaped frame may be formed out of one or more components that are able to support their own weight, and optionally is also able to support fabrics or other materials, e.g., as discussed herein.

However, it should be understood that in some embodiments, the frame need not be a shaped frame. For example, in some cases, the frame need not have a specific or predetermined shape, but may instead be amorphous or flexible, etc. For example, the frame may comprise relatively soft materials, such as soft or deformable plastics. Such non-shaped frames may be useful in some embodiments, e.g., due to the flexibility in being shaped for a particular application, or to minimize injury if the subject comes into contact with the frame.

If a shaped frame is used, it may define a variety of shapes and internal volumes. In some cases, the frame may define a generally rectangular or “box” shaped interior region, although in other embodiments, other shapes are also possible. For instance, the interior region may be spherical, oval, or the like. In some cases, the shaped frame itself defines a suitable interior region, e.g., when positioned above a bed, although in other cases, the shaped frame may be constructed and arranged such that the shaped frame in conjunction with one or fabrics or other materials (e.g., connected to and/or placed on top of the frame) together defines a suitable interior region (e.g., when positioned above a bed).

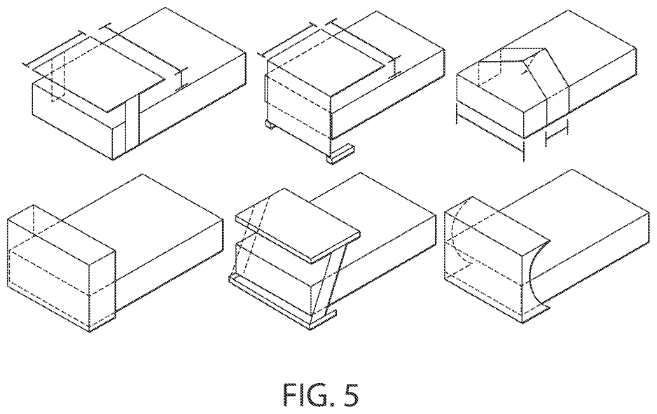

A variety of non-limiting examples of shaped frames are depicted in FIGS. 3-5. In each of these examples, shown in perspective view, the frame would be positioned above a bed, with the subject exiting to the right of the shaped frame. It will be noted that in some of these examples, a space is provided on the right portion of the frame (in the views and orientations shown in these figures) for the subject's body or limbs to exit the interior region defined by the frame. Thus, for example, a frame may have a curved or cut-out portion that allows the legs to be swung in and out without contacting the frame, e.g., due to the shape of the frame, e.g., as shown in last diagram of FIG. 5. Of course, other configurations of shaped frames are also possible in addition to these.

In some embodiments, the shaped frame may include one, two, or more legs or other members that at least partially defines the internal region. However, it should be understood that in other embodiments, the shaped frame may not necessarily have individual legs or other members. In addition, in certain cases, the shaped frame may be adjustable in shape and/or size, e.g., by adjusting one or more of the legs, members, or other components. Those of ordinary skill in the art will be aware of different methods of adjusting the lengths of such components, e.g., using mechanisms that can be screwed together, components that fit inside (and optionally can be locked into position within) other components, or the like.

In some embodiments, the frame may define an interior region having a volume that is at least sufficiently large as to partially or completely contain an extremity. For example, the interior volume may have a largest maximum internal dimension (e.g., a length completely contained within the interior volume of the frame, when in use, for example, positioned on a bed) of no more than 5 ft, no more than 4 ft, no more than 3 ft, no more than 2 ft, or no more than 1 ft (1 ft=0.3048 m). In certain cases, the frame may have an interior volume that is no more than 15 ft³, no more than 12 ft³, no more than 10 ft³, no more than 8 ft³, no more than 6 ft³, no more than 5 ft³, no more than 4 ft³, no more than 3 ft³, no more than 2 ft³, or no more than 1 ft³.

The shaped frame may be formed out of a variety of materials in various embodiments. In some cases, relatively rigid materials may be used, e.g., to ensure that the frame is able to maintain a certain shape. However, in other cases, relatively softer materials can be used, e.g., to prevent or minimize injury if a subject comes into contact with the shaped frame. Non-limiting examples of suitable materials include a metal (e.g., stainless steel, aluminum such as extruded aluminum, etc.), plastic (e.g., polyvinyl chloride, polycarbonate, polystyrene, polyethylene, etc.), wood, rubber (e.g., synthetic or natural), fiberboard (e.g., MDF or medium density fiberboard), or the like.

In some embodiments, a fabric or other material may be positioned so as to cover at least a portion of the interior region. In some cases, the fabric may help to define the interior region, e.g., when placed on a frame. A variety of fabrics or other materials may be used in various embodiments. Non-limiting examples of fabrics include curtains, towels, or bedding materials, such as sheets or blankets. Other examples include plastic or rubber sheets, etc. These may be attached or affixed to the frame, and/or may remain separate from the frame. For example, the fabric or other material may be loosely draped on top of the frame, and is thereby kept from coming into contact with the subject's extremities due to the shape of the frame. The fabric or other material may be sold as part of the device, or may be separately provided by a user of the device, etc.

The fabric or other material may be positioned so as to partially or completely enclose the interior region of the frame. In some cases, the fabric may be positioned to enclose the interior region of the frame except for one or more openings where the extremities or limbs of the subject can pass through. The fabric may come into contact with the subject at that point, or be positioned so as to avoid contact, in different embodiments.

In certain embodiments, the device may be positioned or placed relative to the subject, or the bed (or other article of furniture) for the subject, and optionally held in position. For example, a variety of methods may be used to maintain the position of the interior region around a portion of a subject, e.g., an extremity or a limb, for example, such that the subject does not come into contact with the device.

For example, in one set of embodiments, the frame may be simply placed on the bed, and may stay in place due to its own weight, and/or by partially deforming the bed due to its weight, e.g., causing portions of a mattress to partially compress, etc. Thus, the frame may be supported by portions of the bed (or other article of furniture), e.g., on the mattress, and/or on bedding materials (e.g., on pillows, sheets, blankets, etc.) that may be present on the bed. An example of such an embodiment is shown in FIG. 2C.

In addition, in another set of embodiments, the frame may be immobilized relative to the bed. For instance, the frame may be immobilized relative to a mattress or a bedframe of the bed. In some cases, the frame may be immobilized using clamps, braces, ties, or other suitable attachments. As a non-limiting example, the frame may be immobilized using a base that is positioned between two mattresses, or between a mattress and a bedframe, e.g., as is shown in FIG. 2B.

In certain embodiments, however, the frame may not necessarily rely on the bed to remain in position. For example, the frame may be held in place relative to the floor or other solid surface, or to a ceiling in some cases (e.g., the frame may be suspended from a ceiling). As a non-limiting example, the device may include a stand which supports the frame at a height over the stand. This may be a height that allows a bed to be present under the frame. The frame may thus be positioned at a suitable height above the stand or above the floor, e.g., at a height of at least 1 ft, at least 1.2 ft, at least 1.4 ft, at least 1.5 ft, at least 1.6 ft, at least 1.8 ft, at least 2 ft, at least 2.2 ft, at least 2.4 ft, at least 2.5 ft, at least 2.6 ft, at least 3 ft, at least 3.2 ft, at least 3.4 ft, at least 3.5 ft, at least 3.6 ft, at least 3.8 ft, at least 4 ft, etc., and/or at a height of no more than 4 ft, no more than 3.8 ft, no more than 3.6 ft, no more than 3.5 ft, no more than 3.4 ft, no more than 3.2 ft, no more than 3 ft, no more than 2.8 ft, no more than 2.6 ft, no more than 2.5 ft, no more than 2.4 ft, no more than 2.2 ft, no more than 2 ft, no more than 1.8 ft, no more than 1.6 ft, no more than 1.5 ft, no more than 1.4 ft, no more than 1.2 ft, no more than 1 ft, etc. Combinations of any of these are possible, e.g., the frame may be positioned at a height of between 1.5 ft and 3 ft, e.g., relative to a stand or relative to the floor, etc. One non-limiting example of such an embodiment is shown in FIG. 1A, with interior region 35 being defined between shaped frame 30 and mattress 22.

In one aspect, air may be directed into the interior region of the device, e.g., to ventilate the interior region, and/or to regulate or control the temperature therein. As discussed herein, the air may be, for example, cooled air produced by a cooling system, and/or heated air from a heating system. In addition, in some instances, the air may be ambient air.

A variety of distributors may be used to distribute the air within the interior region. For example, air may be introduced into the interior region from one or more outlets. The outlets may be positioned with one or more legs, members, conduits, ducts, manifolds, etc., which may be positioned in any suitable location so as to direct air at least partially into the interior region. Non-limiting examples of distributors are shown in FIG. 4, illustrating distributors comprising pipes or ducts with various outlets. In addition, a planar distributor is illustrated in FIG. 1B.

The distributor may be formed as part of the frame, and/or the distributor may be separate from the frame. For instance, in one embodiment, a frame may contain a distributor, e.g., in a leg or other member. The distributor may contain one or more conduits, manifolds, tubes, ducts, pipes, or the like, e.g., for receiving air from a source of air and distributing the air into the interior region. There may be one or more outlets or openings in the distributor to allow air to enter the interior region. In some cases, the air may be uniformly distributed within the interior region by the distributor, although in other case, the air may be non-uniformly distributed. For example, the air may be directed towards the center of the interior region, directed at a specific location (e.g., an extremity of a subject), directed towards one or more sides of the interior region (e.g., forming a “curtain” of air), etc. In some cases, a fan or other device may be used to facilitate the distribution of air within the interior region by the distributor, although in other cases, no such device is used, e.g., the air may be distributed passively.

In some aspects, one or more conduits, manifolds, tubes, ducts, pipes, or the like may be used to direct air from a source of air to the distributor. The source of air may be, for example, a cooling system able to produce cool air, a heating system to produce heated air, a fan or other system able to introduce ambient air (e.g., not heated or cooled), or the like. The source of air may be portable or battery-powered, or in some cases, the source of air can be powered using a common electrical outlet.

The source of air can be positioned in any suitable location within the device. For example, the source of air may be contained within the interior region, or not contained within the interior region. In some cases, for example, the source of air may be positioned on the floor or on a base, e.g., as is shown in the example of FIG. 1A. The source of air may be immobilized relative to the frame, or may be separate from the frame. In some cases, a separate source of air may be useful for making devices that are easier to reconfigure, e.g., for different beds and bedroom environments. The source of air may be attached to the distributor using one or more conduits, manifolds, tubes, ducts, pipes, etc., as noted above.

In one set of embodiments, the source of air may be able to cool the air, e.g., using a suitable cooling system. Non-limiting examples of cooling systems able to produce cool air include, but are not limited to, air conditioner, a refrigeration unit, ice, dry ice, a heat pump, an electric cooler (e.g., a Peltier cooler or a thermoelectric cooler), or the like. In yet another embodiment, a mattress (e.g., within the bed) may be used as a suitable cooling system to cool the air.

In addition, in some embodiments, the source of air may be able to heat the air, e.g., using a suitable heating system. The heating system and the cooling system may be separate systems, or may be integrated together within a common source of air. Non-limiting examples of heating systems include, but are not limited to, electrical heaters, gas-based heaters, infrared lights, etc. In yet another embodiment, a mattress (e.g., within the bed) may be used as a suitable heating system to heat the air.

In some cases, the same system may be able to both heat and cool the air, e.g., as desired. For example, in one embodiment, a Peltier system may be used to either cool or heat the air, depending on which way current flows through the Peltier. In some cases, a microcontroller may be used control of the current within the Peltier system, e.g., as controlled by a user. As another non-limiting example, a mattress may be provided that can both heat or cool, and thus the mattress may be able to both heat and cool the air as desired.

In certain cases, the temperature of the interior may be controlled, e.g., as discussed herein. For example, the temperature of the interior space may be between 55° F. and 70° F. In some embodiment, the temperature may be between 55° F. and 60° F., between 55° F. and 65° F., between 60° F. and 70° F., or between 65° F. and 70° F. Other temperature ranges, e.g., below 55° F. or above 70° F. are also possible in certain cases.

In some embodiments, the source of air may be controlled, e.g., using a thermostat or other suitable control system, to regulate or control the temperature of the air that is produced. For instance, in one set of embodiments, the device may contain a thermometer or a temperature sensor (e.g., a thermistor, a thermocouple, a resistance temperature detector, etc.) that is able to determine an air temperature (e.g., of the interior region). Optionally, the thermometer or a temperature sensor may be able to pass such temperature information to a control system, e.g., that is able to regulate or control the temperature of the air that is produced by the source of air in order to control the temperature within the interior region. A variety of systems and methods for determining a temperature and determining a suitable control response will be known to those of ordinary skill in the art. In addition, in some cases, the amount of air entering the interior region may be controlled, at least in part, based on such temperature information, e.g., to ensure that the temperature within the interior region is at or near a specific value or setpoint, or at least within a certain range. Such temperatures may be predetermined, or controlled by a user of the device, etc. For example, in response to a temperature reading that is too hot or too cold, relative to a setpoint or a set temperature range, the control system may increase or decrease (or turn on or off) the production of air by the source of air, activate or alter the amount of heating or cooling by a heating or cooling system, etc.

In certain embodiments, a variety of techniques may be used to control the device, e.g., the temperature, airflow rate, concentration of aromatics, or the like. In some cases, a microcontroller may be present to control the device, and optionally, accept control from a user of the device. As a non-limiting example, the temperature may be pre-programmed, or controllable, by a user. In addition, in some embodiments, a user may be able to control the device using wireless networks such as WiFi, Bluetooth, Zigbee, RF, RFID, Sigfox, IR, ultrasound, radio, or the like.

In addition, in some embodiments, one or more aromatic substances may be added to the air. For example, such substances may be present within the source of air, or the air may be passed over a suitable source of such aromatics. The aromatics may be added in a liquid or a solid form, etc. For instance, in certain cases, the aromatics within the source of aromatics may be volatile, and can become gaseous and enter the air flow. In some cases, wicks or reeds may be used to facilitate the transfer of aromatics. In some cases, an aeration device or a fan may be used to facilitate volatilization of aromatics. However, it should be understood that in some embodiments, the aromatics may be released into the interior region independently of the source of air, e.g., using a separate airflow system. For example, an aeration device or a fan may be used to introduce the aromatics into the interior region.

As another example, a nebulizer may be used to introduce the aromatics. For instance, the nebulizer may be able to use oxygen, air (e.g., compressed air), ultrasound, water-based misting, or the like to break up a liquid into aerosol droplets. In some case, the nebulizer may be removable. The nebulizer may be positioned to introduce the aromatics into the air flow, separately introduce into the interior region, etc.

Non-limiting examples of such aromatic substances include, but are not limited to, essential oils, fragrances, perfumes, ethanol, aromas, or the like. The aromatic substances may include compounds such as esters, linear terpenes, cyclic terpenes, aromatics, amines, alcohols, aldehydes, or the like. For example, the aromatic substance may include oils and scents such as chamomile, lavender, rose, ylang-ylang, basil, laurel, lemongrass, peppermint, rosemary, black pepper, nutmeg, orange, bergamot, lemon, tangerine, anise, cumin, cardamom, fennel, allspice, juniper, cinnamon, cassia, sassafras, cedarwood, camphor, sandalwood, ginger, vetiver, turmeric, myrrh, frankincense, or the like.

In some cases, the molecular weight may be less than about 350 or less than about 300. The aromatic substances may be naturally-occurring, artificially synthesized, or the like. These may be added, for example, to soothe or calm the subject, to freshen the air, to kill pathogens within the air, or the like. In addition, in some cases, the aromatic substances may be selected to produce a particular scent, such as strawberry, vanilla, ocean, or the like.

The following are each incorporated herein by reference in its entirety: U.S. Provisional Patent Application Ser. No. 63/289,763, filed Dec. 15, 2021, entitled “Systems and Methods for Regulating Temperatures around Extremities”; and of U.S. Provisional Patent Application Ser. No. 63/335,812, filed Apr. 28, 2022, entitled “Systems and Methods for Regulating Temperatures around Extremities.”

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A device for regulating temperature around an extremity of a subject, the device comprising:
a structure comprising a shaped frame defining an interior region having a volume at least sufficient to contain an extremity of a subject, wherein the volume is no more than 10 ft³;
a cooling system able to produce cooled air; and
a distributor positioned to distribute the cooled air from the cooling system into the interior region of the shaped frame.

2. The device of claim 1, further comprising a conduit connecting the cooling system to the distributor.

3. The device of any one of claim 1 or 2, further comprising an attachment constructed and arranged to immobilize the shaped frame on a bed.

4. The device of claim 3, wherein the attachment comprises a clamp.

5. The device of any one of claim 3 or 4, wherein the attachment is constructed and arranged immobilize the shaped frame on a mattress of the bed.

6. The device of any one of claims 1-5, wherein the attachment is constructed and arranged immobilize the shaped frame on a bedframe of the bed.

7. The device of any one of claims 1-6, wherein the structure further comprises a stand positioning the shaped frame at a height of between 1.5 ft and 3 ft over a bottom of the stand.

8. The device of any one of claims 1-7, wherein the structure further comprises fabric covering at least a portion of the interior region.

9. The device of any one of claims 1-8, wherein the interior region has a maximum dimension of no more than 2 ft.

10. The device of any one of claims 1-9, wherein the shaped frame comprises at least one leg member defining the interior region.

11. The device of any one of claims 1-10, wherein the shaped frame is constructed and arranged to rest upon a bed to define the interior region.

12. The device of any one of claims 1-11, wherein the shaped frame comprises metal.

13. The device of any one of claims 1-12, wherein the shaped frame comprises aluminum.

14. The device of any one of claims 1-13, wherein the shaped frame comprises plastic.

15. The device of any one of claims 1-14, wherein the shaped frame is adjustable.

16. The device of any one of claims 1-15, further comprising a temperature sensor positioned to determine a temperature of the interior region.

17. The device of claim 16, further comprising a thermostat constructed and arranged to control the cooling system in response to the temperature of the interior region.

18. The device of any one of claims 1-17, wherein the cooling system comprises a fan.

19. The device of any one of claims 1-18, wherein the cooling system comprises an air conditioner.

20. The device of any one of claims 1-19, wherein the cooling system comprises a thermoelectric cooler.

21. The device of any one of claims 1-20, wherein the cooling system comprises a Peltier cooler.

22. The device of any one of claims 1-21, wherein the distributor comprises a fan.

23. The device of any one of claims 1-22, wherein the distributor comprises a conduit having one or more openings directed at the interior region.

24. The device of any one of claims 1-23, further comprising a source of aromatics configured to distribute an aromatic into the cooled air.

25. The device of claim 24, wherein the source of aromatics comprises a liquid in a container.

26. The device of any one of claim 24 or 25, wherein the source of aromatics comprises a nebulizer.

27. The device of any one of claims 24-26, wherein the source of aromatics comprises an essential oil.

28. The device of any one of claims 24-27, wherein the source of aromatics comprises lavender oil.

29. A device for regulating temperature around an extremity of a subject, the device comprising:
a structure comprising a shaped frame defining an interior region;
a brace immobilizing the shaped frame on a mattress or a bedframe;
a cooling system able to produce cooled air; and
a distributor for distributing the cooled air from the cooling system into the interior region of the shaped frame.

30. The device of claim 29, further comprising a conduit connecting the cooling system to the distributor.

31. The device of any one of claim 29 or 30, further comprising an attachment constructed and arranged to immobilize the shaped frame on a bed.

32. The device of claim 31, wherein the attachment comprises a clamp.

33. The device of claim 31 or 32, wherein the attachment is constructed and arranged immobilize the shaped frame on a mattress of the bed.

34. The device of claim 31-33, wherein the attachment is constructed and arranged immobilize the shaped frame on a bedframe of the bed.

35. The device of any one of claims 29-34, wherein the structure further comprises a stand positioning the shaped frame at a height of between 1.5 ft and 3 ft over a bottom of the stand.

36. The device of any one of claims 29-35, wherein the structure further comprises fabric covering at least a portion of the interior region.

37. The device of any one of claims 29-36, wherein the shaped frame has a volume at least sufficient to contain an extremity of a subject.

38. The device of any one of claims 29-37, wherein the shaped frame has a volume of no more than 10 ft³.

39. The device of any one of claims 29-38, wherein the interior region has a maximum dimension of no more than 2 ft.

40. The device of any one of claims 29-39, wherein the shaped frame comprises at least one leg member defining the interior region.

41. The device of any one of claims 29-40, wherein the shaped frame is constructed and arranged to rest upon a bed to define the interior region.

42. The device of any one of claims 29-41, wherein the shaped frame comprises metal.

43. The device of any one of claims 29-42, wherein the shaped frame comprises aluminum.

44. The device of any one of claims 29-43, wherein the shaped frame comprises plastic.

45. The device of any one of claims 29-44, wherein the shaped frame is adjustable.

46. The device of any one of claims 29-45, further comprising a temperature sensor positioned to determine a temperature of the interior region.

47. The device of claim 46, further comprising a thermostat constructed and arranged to control the cooling system in response to the temperature of the interior region on.

48. The device of any one of claims 29-47, wherein the cooling system comprises a fan.

49. The device of any one of claims 29-48, wherein the cooling system comprises an air conditioner.

50. The device of any one of claims 29-49, wherein the cooling system comprises a thermoelectric cooler.

51. The device of any one of claims 29-50, wherein the cooling system comprises a Peltier cooler.

52. The device of any one of claims 29-51, wherein the distributor comprises a fan.

53. The device of any one of claims 29-52, wherein the distributor comprises a conduit having one or more openings directed at the interior region.

54. The device of any one of claims 29-53, further comprising a source of aromatics configured to distribute an aromatic into the cooled air.

55. The device of claim 54, wherein the source of aromatics comprises a nebulizer.

56. The device of any one of claim 54 or 55, wherein the source of aromatics comprises an essential oil.

57. The device of any one of claim 54 or 56, wherein the source of aromatics comprises lavender oil.

58. A method for regulating temperature around an extremity of a subject, the method comprising:
providing a structure comprising a shaped frame defining an interior region, a fabric covering at least portion of the interior region, and a distributor positioned to distribute air into the interior region of the shaped frame;
positioning the structure and the subject relative to each other such that the subject has at least one extremity positioned within the interior region, wherein the at least one extremity does not contact the fabric, and the shaped frame does not surround an entire limb of the subject;
producing cooled air in a cooling system; and
distributing the cooled air at the extremity within the interior region of the shaped frame using the distributor.

59. A device for regulating temperature around an extremity of a subject, the article comprising:
a structure comprising a shaped frame defining an interior region;
a stand positioning the shaped frame at a height of between 1.5 ft and 3 ft over a bottom region of the stand;
a cooling system able to produce cooled air; and
a distributor for distributing the cooled air from the cooling system into the interior region of the shaped frame.

60. A method, comprising:
providing a structure comprising a shaped frame defining an interior region and having a fabric covering;
positioning the structure and the subject relative to each other such that the subject has at least one extremity positioned within the interior region, wherein the at least one extremity does not contact the fabric, and the shaped frame does not surround an entire limb of the subject; and
distributing cooled air at the extremity within the interior region of the shaped frame using a distributor immobilized relative to the shaped frame.
